(19) United States
(12) Patent Application Publication
(10) Pub. No.: US 2004/0131995 A1
(43) Pub. Date: Jul. 8, 2004

(54) Process for the preparation of a polymerizable dental composition

(76) Inventors: Joachim E. Klee, Radolfzell (DE); Uwe Walz, Konstanz (DE); Andreas Facher, Konstanz (DE); Christoph Weber, Konstanz (DE); Rolf Mulhaupt, Freiburg (DE); Holger Frey, Emmendingen (DE); Ekkehard Muh, Rheinfelden (DE)

Correspondence Address: Douglas J. Hura, Esquire, DENTSPLY INTERNATIONAL INC., 570 West College Avenue, York, PA, 17405, US

(21) Appl. No.: 10/617,503

(22) Filed: Jul. 11, 2003

(30) Foreign Application Priority Data
Jan. 15, 2001 (DE) 10101537.2
Jan. 15, 2002 (WO) PCT/EP02/00338

(51) Int. Cl. A61F 2/00; A61C 5/00

(52) U.S. Cl. 433/217.1; 523/115

(57) ABSTRACT

A process for the preparation of a polymerizable dental composition comprising the steps of

(a) preparing a liquid mixture comprising

(i) 1 to 99% w/w of a hybrid monomer component containing at least one hybrid monomer compound having one hydrolysable siloxane group and at least one polymerizable organic moiety, and

(ii) 99 to 1% w/w of a monomer component polymerizable with the polymerizable organic moiety of the hybrid monomer compounds; and

(b) adding at least a stoichiometrically sufficient amount of water to the mixture to hydrolyse the hydrolysable siloxane group of the hybrid monomer compound and to form spherical polymerizable nanoparticles having an average particle size of from 1 to 100 nm dispersed in the monomer component, whereby the nanoparticles have a structure with Si—O—Si bonds and peripherally exposed polymerizable organic moieties.

The present invention relates to a process for the preparation of a polymerizable dental composition. In particular, the present invention relates to a process for the preparation of a polymerizable dental composition containing specific small particles. Moreover, the present invention relates to a polymerizable dental composition obtainable by the claimed process.

The synthesis of hydrolysable siloxane monomers containing polymerizable moieties is disclosed in U.S. Pat. No. 6,124,491. Hydrolysis of these monomers leads to polymerizable polycondensates.

The incorporation of polymerizable polysiloxanes into polymerizable dental compositions for improving physical properties of the polymerised compositions is known from DE-A 199 03 177.

DE-A 198 16 148 and DE-A 198 47 635 disclose polymerizable dental compositions comprising a polymerizable component and organopolysiloxane particles. The particles are sperical microgels having an average particle size of 5 to 200 nm, each consisting of a single crosslinked molecule. The polymerizable dental compositions are prepared by preparation of the particles in a polar solvent and subsequent mixing of the isolated particles with a polymerizable base component. The preparation of the particles is a complicated operation requiring multiple reaction steps including the hydrolysis of suitable siloxane precursors, the saturation of remaining condensable groups with monofunctional triorganosilyl groups for avoiding condensation between particles, and the isolation of the particles from a colloidal suspension system. EP-B1 0 744 432 also discloses such generic particles and processes for their preparation.

The particles known from the prior art are problematic. It is difficult to handle the particles prepared according to the prior art processes since they tend to agglomerate when isolated from the reaction mixture in which they are formed. Agglomeration results in the formation of aggregates which increase the viscosity of a dental composition and which may deteriorate the optical properties when the size of the aggregates is in the order of the wave-length of visible light. Moreover, since the formation of aggregates is a thermodynamically favoured process, the redispersion of the particles in polymerizable monomers requires extremely energy and time-consuming processes.

Therefore, it is the problem of the present invention to provide a process for the preparation of a polymerizable dental composition containing well-defined nanoparticles whereby the process does not involve complicated, energy- and time-consuming reaction-steps.

Accordingly, the present invention provides a process for the preparation of a polymerizable dental composition comprising the steps of

(a) preparing a liquid mixture comprising

(i) 1 to 99% w/w of a hybrid monomer component containing at least one hybrid monomer compound having one hydrolysable siloxane group and at least one polymerizable organic moiety, and

(ii) 99 to 1% w/w of a monomer component polymerizable with the polymerizable organic moiety of the hybrid monomer compounds; and

(b) adding at least a stoichiometrically sufficient amount of water to the mixture to hydrolyse the hydrolysable siloxane group of the hybrid monomer compound and to form spherical polymerizable nanoparticles having an average particle size of from 1 to 100 nm dispersed in the monomer component, whereby the nanoparticles have a structure with Si—O—Si bonds and peripherally exposed polymerizable organic moieties.

The present invention provides a homogeneous mixture of spherical polymerizable nanoparticles in a monomer component, such as a reactive diluent. The term nanoparticles in this specification is used for particles having an average particle size of from 1 to 100 nm.

The nanoparticles are formed in situ in a low polarity monomer component whereby it is not necessary to isolate and redisperse the nanoparticles in a dental composition. Moreover, the particles according to the invention may be used without further saturation of remaining condensable groups with monofunctional triorganosilyl groups for avoiding condensation between particles. Thereby, the process of the invention provides a dental composition in a one-pot reaction without the need for complicated, energy- and time-consuming reaction-steps. The nanoparticles are dispersed in the monomer component in a stable and homogeneous manner whereby agglomeration of the nanoparticles to aggregates is avoided (compare example 7 and comparative examples 1 and 2 in Table 3).

It was found that, surprisingly, the hydrolysis of the hydrolysable siloxane groups in a polymerizable monomer component, preferably of low polarity, leads to particles having a narrow particle size distribution and a well-defined structure with Si—O—Si bonds and peripherally exposed polymerizable organic moieties. The nanoparticles may subsequently be copolymerised with the polymerizable monomer component whereby a polymerised matrix of the monomer component is formed wherein the dispersed nanoparticles are cross-linked to the matrix. The incorporation of the nanoparticles into the polymerised matrix of the monomer component according to the invention provides a cured dental composition having increased strength and decreased polymerisation shrinkage, while the dental composition has the same or only slightly increased viscosity, preferably less than 10%, as compared to the same composition not containing nanoparticles.

Preferably, the nanoparticles formed according the invention have an average particle size of from 1 to 20 nm, most preferably of from 1 to 5 nm. The size of the nanoparticles may be controlled by the choice of the type and amount of the hybrid monomer component as well as the presence of further cohydrolysable components.

The process according to the invention comprises the step of preparing a liquid mixture comprising 1 to 99% w/w of a hybrid monomer component containing one or more hybrid monomer compounds having a polymerizable organic moiety and a hydrolysable group, and 99 to 1% w/w of a monomer component polymerizable with the polymerizable organic moiety of the hybrid monomer compounds.

In one embodiment, the process according to the invention comprises the step of preparing a liquid mixture comprising 1 to 50% w/w of a hybrid monomer component containing one or more hybrid monomer compounds having a polymerizable organic moiety and a hydrolysable group, and 99 to 50% w/w of a monomer component polymerizable with the polymerizable organic moiety of the hybrid monomer compounds. Preferably, the mixture comprises 90% w/w or more of the monomer component, more preferably 70% w/w or more of the monomer component. According to this embodiment, a dental composition having a low content of nanoparticles is formed.

In another embodiment, the process according to the invention comprises the step of preparing a liquid mixture comprising 50 to 99% w/w of a hybrid monomer component containing one or more hybrid monomer compounds having a polymerizable organic moiety and a hydrolysable group, and 50 to 1% w/w of a monomer component polymerizable with the polymerizable organic moiety of the hybrid monomer compounds. Preferably, the mixture comprises 30% w/w or less of the monomer component, more preferably 10% w/w or less of the monomer component. According to this embodiment, a dental composition having a high content of nanoparticles is formed.

The hybrid monomer compounds used in the process of the present invention preferably contain a hydrolysable siloxane group according to the following formula (I):

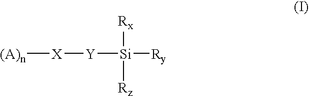

wherein

A is a polymerizable moiety, preferably an acrylate or methacrylate group;

Rₓ, R_{y}, R_{z}

which may be the same or different independently represent a substituted or unsubstituted C₁ to C₁₈ alkoxy, C₅ to C₁₈ cycloalkoxy, a C₅ to C₁₅ aryloxy, C₂ to C₁₈ acyloxy or halogen;

X is a nitrogen atom or a substituted or unsubstituted C₁ to C₁₈ alkylene, C₁ to C₁₈ oxyalkylene or C₁ to C₁₈ carboxyalkylene group;

Y is a substituted or unsubstituted C₁ to C₁₈ alkylene, C₁ to C₁₈ oxyalkylene, C₅ to C₁₈ cycloalkylene, C₅ to C₁₈ oxycycloalkylene, C₅ to C₁₅ arylene, or C₅ to C₁₅ oxyarylene or heteroarylene group, or a urethane, —O—CONH— or a thiourethane —OCSNH— linking moiety; and

n is an integer of 1 to 10, preferably of from 1 to 5.

The group A defined as a polymerizable moiety may be any moiety containing a multiple bond capable of undergoing radical polymerisation. Preferably the multiple bond is a carbon-carbon double bond. Preferred moieties for A are an acrylate or methacrylate group.

Rₓ, R_{y}, R_{z} may be the same or different. Rₓ, R_{y}, R_{z} are chosen so as to provide hydrolysable leaving groups allowing or facilitating hydrolysis and crosslinking of the hybrid monomer component to form intermolecular Si—O—Si bonds in admixture with a monomer component such as a reactive diluent.

Rₓ, R_{y}, R_{z} defined as C₁ to C₁₈ alkoxy may be straight-chain or branched radicals, for example methoxy, ethoxy, n-propoxy, isopropoxy, isobutoxy, sec-butoxy and tert-butoxy as well as radicals of higher alkanols such as the different isomers of pentyloxy, hexyloxy, heptyloxy, octyloxy, nonyloxy, decyloxy, undecyloxy, or dodecyloxy, tridecyloxy, tetradecyloxy, pentadecyloxy, hexadecyloxy, heptadecyloxy, or octadecyloxy.

Rₓ, R_{y}, R_{z} defined as C₅ to C₁₈ cycloalkoxy are mono or polycyclic radicals containing 5 to 18 ring-carbon atoms, e.g. cyclopentyloxy, cyclohexyloxy, cycloheptyloxy or cyclooctyloxy.

Rₓ, R_{y}, R_{z} defined as a C₅ to C₁₅ aryloxy can be, for example, phenoxy, tolyloxy, indenyloxy, and napthyloxy.

Rₓ, R_{y}, R_{z} defined as C₂ to C₁₈ acyloxy, may be a straight or branched radical wherein an acyl group is bonded via an oxygen atom. “Acyl” means an HCO— or (alkyl) CO— group in which the alkyl group is a straight-chain or branched radical, for example methyl, ethyl, n-propyl, isobutyl, sec-butyl and tert-butyl as well as the different isomers of pentane, hexane, heptane and octane. Exemplary acyloxy groups include formyloxy, acetyloxy, propanoyloxy, 2-methylpropanoyloxy, butanoyloxy and palmitoyloxy.

Rₓ, R_{y}, R_{z} defined as halogen may be chlorine, bromine or iodine, preferably chlorine or bromine.

The expression “substituted” applied to Rₓ, R_{y}, R_{z} means that the C₁ to C₁₈ alkoxy, C₅ to C₁₈ cycloalkoxy, a C₅ to C₁₅ aryloxy, or C₂ to C₁₈ acyloxy groups may be substituted by, preferably from 1 to 5, identical or different substituents selected from C₁ to C₆ alkoxy groups, C₁ to C₆ alkylthio groups, C₁ to C₆ alkylamino groups, di-(C₁ to C₆ alkyl)amino groups, halogen atoms such as fluorine, chlorine or bromine, C₁ to C₆ acyloxy groups, or C₁ to C₆ acylamido groups. Preferred substituents are C₁ to C₆ alkoxy groups, C₁ to C₆ alkylthio groups, C₁ to C₆ alkylaminogroups, and di-(C₁ to C₆alkyl)amino groups.

X defined as C₁ to C₁₈ alkylene means the straight-chain groupings —(CH₂)ₐ—, wherein a=1 to 18, i.e. for example methylene, ethylene, n-propylene, as well as the branched bifunctional groupings of propene, butene, pentene, hexene, heptene, octene and higher homologues, whereby the alkylene group may be further substituted by 1 to 9 moieties of group A as defined above, such as acryloxy groups or methacryloxy groups.

X defined as C₁ to C₁₈ oxyalkylene means the straight-chain groupings —O(CH₂)ₐ—, wherein a=1 to 18, i.e. for example oxymethylene, oxyethylene, oxy-n-propylene, as well as the branched bifunctional groupings of oxypropene, oxybutene, oxypentene, oxyhexene, oxyheptene, oxyoctene and higher homologues, whereby the oxyalkylene group may be further substituted by 1 to 9 moieties of group A as defined above-such as acryloxy groups or methacryloxy groups.

X defined as C₁ to C₁₈ carboxyalkylene means the straight-chain groupings —OCO(CH₂)ₐ—, wherein a=1 to 18, i.e. for example carboxymethylene, carboxyethylene, carboxy-n-propylene, as well as the branched bifunctional groupings of carboxypropene, carboxybutene, carboxypentene, carboxyhexene, carboxyheptene, carboxyoctene and higher homologues, whereby the carboxyalkylene group may be further substituted by 1 to 9 moieties of group A as defined above such as acryloxy groups or methacryloxy groups.

Y defined as C₁ to C₁₈ alkylene means the straight-chain groupings —(CH₂)ₐ—, wherein a=1 to 18, i.e. for example methylene, ethylene, n-propylene, as well as the branched bifunctional groupings of propene, butene, pentene, hexene, heptene, octene and higher homologues.

Y defined as C₁ to C₁₈ oxyalkylene means the straight-chain groupings —O(CH₂)ₐ—, wherein a=1 to 18, i.e. for example oxymethylene, oxyethylene, oxy-n-propylene, as well as the branched bifunctional groupings of oxypropene, oxybutene, oxypentene, oxyhexene, oxyheptene, oxyoctene and higher homologues.

Y defined as C₅ to C₁₈ oxycycloalkylene means cyclic radicals containing 5 to 18 ring-carbon atoms, e.g. of oxycyclopentane, oxycyclohexane, oxycycloheptane and oxycyclooctane groupings.

Y defined as C₅ to C₁₅ arylene may be, for example, phenylene, tolylene, pentalinylene, indenylene, napthylene, azulinylene and anthrylene.

Y defined as C₅ to C₁₈ oxyarylene may be the above arylene groups connected by an oxygen atom.

Y defined as heteroarylene group means mono- or polycyclic aromatic compounds containing one or more atoms other than carbon in the ring.

The expression “substituted” applied to Y means that the C₁ to C₁₈ alkylene, C₁ to C₁₈ oxyalkylene, C₅ to C₁₈ cycloalkylene, C₅ to C₁₈ oxycycloalkylene, C₅ to C₁₅ arylene, or C₅ to C₁₅ oxyarylene or heteroarylene groups are substituted by from 1 to 5 identical or different substituents selected from C₁ to C₆ alkoxy groups, C₁ to C₆ alkylthio groups, C₁ to C₆ alkylamino groups, di-(C₁ to C₆ alkyl)amino groups, halogen atoms such as fluorine, chlorine or bromine, C₁ to C₆ acyloxy groups, or C₁ to C₆ acylamido groups. Preferred substituents are C₁ to C₆ alkoxy groups, C₁ to C₆ alkylthio groups, C₁ to C₆ alkylaminogroups, and di-(C₁ to C₆alkyl)amino groups.

Most preferably, the hybrid monomer compound is a compound of the following formulas 1-10:

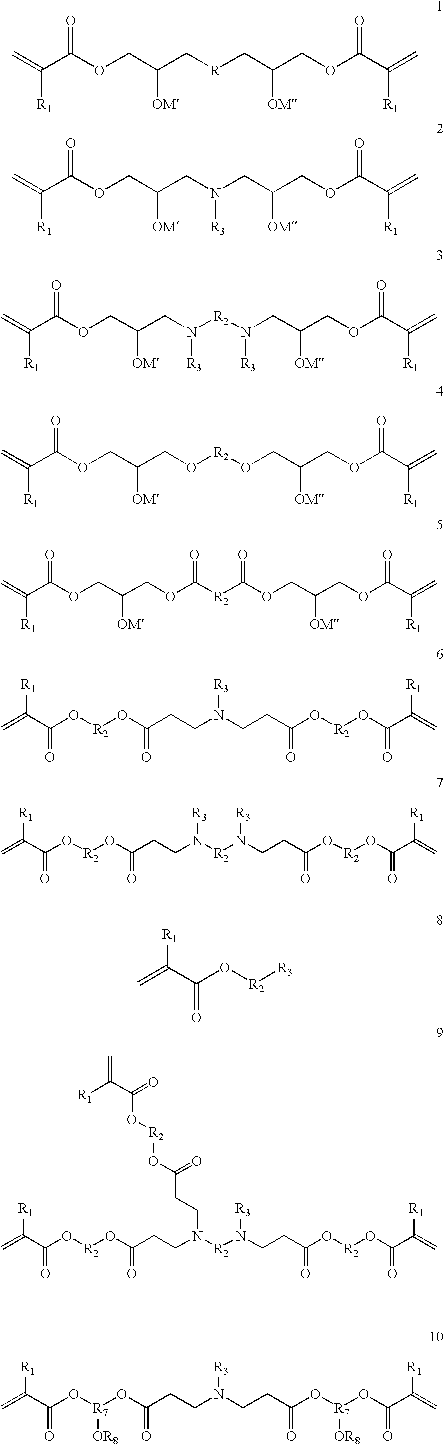

wherein

R is a residue derived from a diepoxide, notably a residue of the following formula

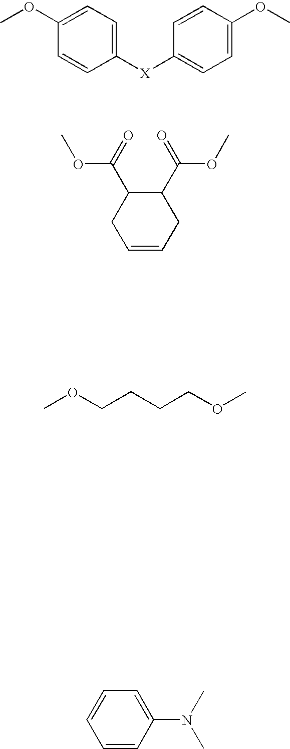

wherein X is C(CH₃)₂, —CH₂—, —O—, —S—, —CO—, or —SO₂—;

R₁ is hydrogen or a substituted or unsubstituted C₁ to C₁₈ alkyl, C₅ to C₁₈ cycloalkyl, C₅ to C₁₈ aryl or heteroaryl group;

R₂ is a divalent substituted or unsubstituted C₁ to C₁₈ alkylene, C₂ to C₁₂ alkenylene, C₅ to C₁₈ cycloalkylene, C₅ to C₁₈ arylene or heteroarylene,

R₃ which may represent the same or different substituents in formula 3 and 7, is a substituted or unsubstituted C₁ to C₁₈ alkyl, C₂ to C₁₂ alkenyl, C₅ to C₁₈ cycloalkyl, C₆ to C₁₂ aryl or C₇ to C₁₂ aralkyl group, or a siloxane moiety represented by one of the following formulae I, II or III

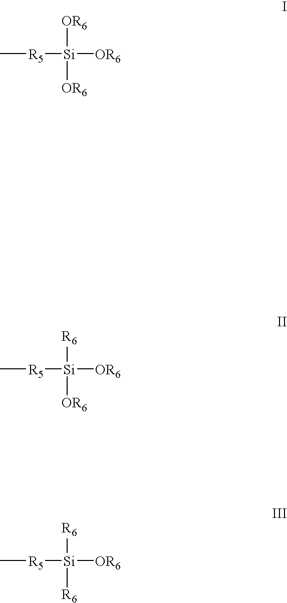

wherein

R₅ is a divalent substituted or unsubstituted C₁ to C₁₈ alkylene, C₂ to C₁₂ alkenylene, C₅ to C₁₈ cycloalkylene, C₅ to C₁₈ arylene or heteroarylene group, preferably CH₂CH₂CH₂,

R₆ is a substituted or unsubstituted C₁ to C₁₈ alkyl, C₂ to C₁₂ alkenyl, C₅ to C₁a cycloalkyl, C₆ to C₁₂ aryl or C₇ to C₁₂ aralkyl group,

R₇ is a substituted or unsubstituted C₁ to C₁₈ alkylene, C₂ to C₁₂ alkenyl, C₅ to C₁₈ cycloalkylene, C₅ to C₁₈ arylene or heteroarylene group,

R₈ is a protecting group for a hydroxyl group, preferably forming an ether, an ester or an urethane group,

M′ and M″

which may represent the same or different substituents, is a siloxane moiety represented by one of the following formulae IV, V or VI, a protecting group for a hydroxyl group, preferably forming an ether, an ester or an urethane group, or hydrogen in case R₃ is a siloxane moiety represented by one of formulae I, II, or III as defined above,

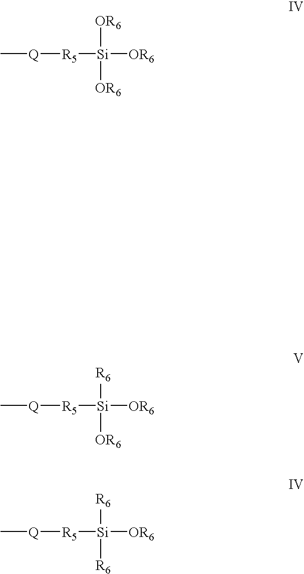

wherein

Q is an ether, an ester, a urethane or thiourethane linking group, and R₅ and R₆ are as defined above.

The above alkyl, alkenyl, cycloalkyl, aralkyl, alkylene, alkenylene and cycloalkylene groups may be staight or branched.

Optional substituents for Rₓ, R_{y}, R_{z}, X, Y, R₁, R₂, R₃, R₅, R₆, and R₇ are selected from of C₁ to C₆ alkoxy groups, C₁ to C₆ alkylthio groups, C₁ to C₆ alkylamino groups, di-(C₁ to C₆ alkyl)amino groups, halogen atoms such as fluorine, chlorine or bromine, C₁ to C₆ acyloxy groups, or C₁ to C₆ acylamido groups. Preferred substituents are C₁ to C₆ alkoxy groups, C₁ to C₆ alkylthio groups, C₁ to C₆ alkylaminogroups, and di-(C₁ to C₆alkyl)amino groups. At least one of these substituents may be present. In case more than one substituent is present, the substituents may be the same or different.

Specific examples of the hybrid monomer compounds are shown by the following formulae 11-12:

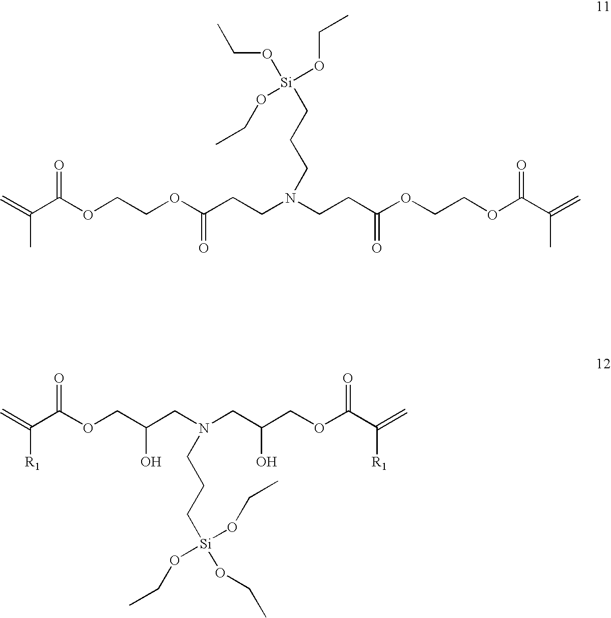

The monomer component polymerizable with the polymerizable organic moiety of the hybrid monomer compounds according to the present invention is preferably selected from mono- or polyfunctional acrylates or methacrylates. Specific examples of the monomer component polymerizable with the polymerizable organic moiety of the hybrid monomer compounds are as follows: methyl methacrylate, ethyleneglycol dimethacrylate, diethyleneglycol dimethacrylate, triethyleneglycol dimethacrylate, 3,(4),8,(9)-dimethacryloyloxymethyltricyclodecane, dioxolan bismethacrylate, vinyl-, vinylen- or vinyliden-, acrylic- or methacrylic substituted spiroorthoesters, spiroorthocarbonates or bicyloorthoesters, glycerin trimethacrylate, trimethylol propane triacrylate, furfurylmethacrylate.

The monomer component polymerizable with the polymerizable organic moiety of the hybrid monomer compounds may be a mixture of the above compounds.

Furthermore, the monomer component polymerizable with the polymerizable organic moiety of the hybrid monomer compounds may be a mixture of the above compounds with other polymerizable monomers such as urethane dimethacrylates like 2,7,7,9,15-pentamethyl-4,13-dioxo-3,14-dioxa-5,12-diaza-hexadecane-1,16-diyl-dimethacrylate (UDMA) or aromatic dimethacrylates such as 2,2-bis-[p-(ù-methacryloyloxy oligo(ethoxy))-phenyl]-propane.

According to the invention, a stoichiometrically sufficient amount of water is added to the mixture of the hybrid monomer component and monomer component to hydrolyse the hydrolysable siloxane groups of the hybrid monomer compounds and to form spherical polymerizable nanoparticles. Water is added in an amount sufficient to hydrolyse all reactive siloxane bonds present in the reaction mixture in the course of the reaction.

The hybrid monomer compounds may be hydrolysed to form polymerizable nanoparticles in the presence of minor amounts of organic solvents such as THF, dioxane, chloroform, toluene, ethyl acetate or acetone.

The hydrolysis of hybrid monomer compounds is carried out in the presence of an acid or base catalyst or under neutral conditions. The hydrolysis is preferably carried out at a temperature of between −20 and +120° C., conveniently at room temperature. The reaction rate of the hydrolysis and formation of nanoparticles may be increased by the addition of ammonium fluoride or hydrogen fluoride.

Furthermore, it is possible to form nanoparticles of mixtures of different hybrid monomers I.

It is possible to form nanoparticles of mixtures of different hybrid monomers I and other hydrolysable siloxane components that contain groups which are able to undergo step-growth such as aminopropyltriethoxy silane, thiopropyltriethoxy silane, 2,3-epoxy propyltriethoxy silane.

Specific examples show that it is possible to form nanoparticles in the presence of other hydrolysable siloxane components that contain no polymerizable groups such as tetraethoxy silane, tetramethoxy silane, monomethyl triethoxy silane, monomethyl trimethoxy silane, dimethyl diethoxy silane, dimethyl dimethoxy silane or tetrachloro-silane. The use of an additional silane compound will usually lead to an increase of the average particle size whereby an increasing amount of the additional silane compound will increase the average particle size of the particles. The cocondensation of the nanoparticles in the presence of silane compounds will provide nanoparticles wherein the silane compounds are predominantly present in the core portion of the particle.

It is possible to form nanoparticles in the presence of metal compounds selected from the group of alkoxides or metal complexes such as metal acetyl acetonates whereby the metals are selected from the group of Ba, Al, La, Ti, Zr, TI, or other transition elements or elements of the lanthanides or actinides. The use of an additional metal compound will usually lead to an increase of the average particle size whereby an increasing amount of the additional metal compound will increase the average particle size of the particles. The cocondensation of the nanoparticles in the presence of metal compounds will provide nanoparticles having wherein the metal compounds are predominantly present in the core portion of the particle.

The dental composition obtainable with the process of the present invention may be used as such. Further process steps may be added to modify the composition obtainable with the process of the invention. Accordingly, the process of the invention may further comprise a step of adding further components to the dental composition obtainable with the process of the present invention as the case requires. Such components include any components commonly used in the dental field for the preparation of a dental composition such as further polymerizable components, fillers, polymerisation initiators and stabilisers.

Specifically, methyl methacrylate, furfuryl methacrylate, polymerizable di- or poly(meth)acrylates may be mentioned as further polymerizable components. Examples for polymerizable di- or poly(meth)acrylate are ethylene glycol dimethacrylate, diethylene glycol dimethacrylate, triethylene glycol dimethacrylate, trimethylol propane triacrylate, 3,(4),8,(9)-dimethacryloyloxymethyltricyclo decane, dioxolan bismethacrylate, and glycerol trimethacrylate.

The fillers may be selected from La₂O₃, ZrO₂, BiPO₄, CaWO₄, BaWO₄, SrF₂, Bi₂O₃, a porous glass or an organic filler, such as polymer granulate, embrittled glass fibres or a combination of organic and/or inorganic fillers or reactive inorganic fillers.

The invention will now be illustrated by the following examples.

### PREPARATION EXAMPLE 1

50.000 g (225.9 mmol) 3-aminopropyl triethoxysilane, 64.218 g (451.7 mmol) 2,3-(epoxypropoxy) methyl methacrylate and 0.1144 g 2,6-di-tert.-butyl-p-cresol were reacted for four hours at 90° C. The obtained methacrylate terminated macromonomer is soluble in organic solvents such as chloroform, DMF and THF. In the IR-spectrum no absorption of epoxide groups at 915 and 3050 cm⁻¹ was observed. New absorptions appeared at 1720 cm₋₁ (ester groups) and 3400 cm₋₁ (OH group). (C₂₃H₄₃O₉NSi), 505.68 g/mol;|_{(23° C.)}=34 mpa*s

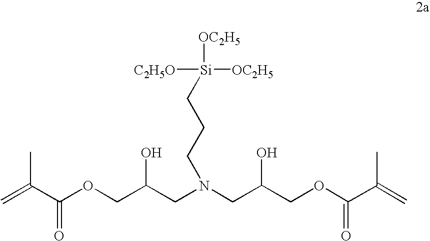

### PREPARATION EXAMPLE 2

50.000 g (278.88 mmol) 3-aminopropyl trimethoxy silan, 79.285 g (557.76 mmol) 2,3-(epoxypropoxy) methyl methacrylate and 0.129 g 2,6-di-tert.-butyl-p-cresol were reacted for four hours at 90° C. The obtained methacrylate terminated macromonomer is soluble in organic solvents such as chloroform, DMF and THF. In the IR-spectrum was observed no absorption of epoxide groups at 915 and 3050 cm₋₁. New absorption's was found at 1720 cm₋₁ (ester groups) and 3400 cm₋₁ (OH group). (C₂₀H₃₇O₉NSi), 463.60 g/mol;|_{(23° C.)}=28 mpa*s

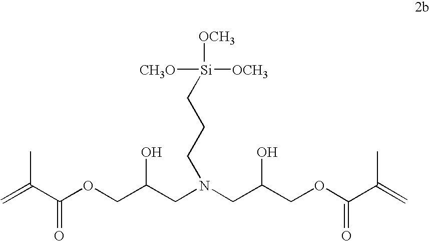

### PREPARATION EXAMPLE 3

Macromonomer 6a:

20.232 g (109.8 mmol) EGAMA, 12.158 g (54.9 mmol) aminopropyl triethoxysilane and 0.032 g BHT were mixed homogeneously and stirred at room temperature for 12 hours for obtaining macromonomer 6a. C₂₇H₄₇NO₁₁Si, 589.75 g/mol; m/z (FAB-MS)=590.

### PREPARATION EXAMPLE 4

Macromonomer 6b

24.574 g (133.42 mmol) EGAMA, 11.960 g (66.71 mmol) aminopropyl trimethoxysilane and 0.037 g BHT were mixed homogeneously and stirred at room temperature for 12 hours for obtaining macromonomer 6b. C₂₄H₄₁NO₁₁Si, 547.24 g/mol; m/z (FAB-MS)=548.

### EXAMPLE 1

### Condensation to Nanoparticles in TGDMA

1.000 g (1.826 mmol) addition product 6b of EGAMA and aminopropyl trimethoxysilane were dissolved in 9.000 g TGDMA. 0.150 g (8.33 mmol) water was added to this solution to obtain a reaction mixture. The reaction mixture was stirred for 14 days at room temperature. The formed particles were found to have an average particle size of 3 nm. The transmission electron microscopic photograph according to FIG. 1 shows the formed nano-scaled particles. In the IR spectrum double bonds of the methacrylate groups were found at 1720 cm₋₁.

### EXAMPLES 2-6

### Condensation to Nanoparticles in TGDMA

Following the same procedure as described in Example 1, further nanoparticles were prepared (Table 1).

TABLE 1
Preparation of nanoparticles in the polymerizable monomer
TGDMA and the viscosity of the resulting condensation mixtures

| | Ratio | | | | |
|---|---|---|---|---|---|
| | hybrid | | m | | |
| | monomer: | m (Addition- | (TGDMA) | m (Water) | Viscosity |
| Example | TGDMA | product) [g] | [g] | [mg] | h [mPas] |
| 1 | 10:90 | 1.000 | 9.000 | 99 | 12 |
| 2 | 30:70 | 3.000 | 7.000 | 296 | 25 |
| 3 | 50:50 | 5.000 | 5.000 | 494 | 61 |
| 4 | 70:30 | 7.000 | 3.000 | 691 | 187 |
| 5 | 90:10 | 9.000 | 1.000 | 888 | 657 |
| 6 | 95:5 | 9.500 | 0.500 | 934 | 1193 |

Nanoparticle solutions 1, 3 and 5 were mixed with 2,2-Bis-[p-(2-hydroxy-3-methacryloyloxypropoxy)phenyl]propane in a ratio of 30/70 wt.-% each. Shrinkage and conversion (DSC) of the mixtures were compared with Bis-GMA/TGDMA (30/70) wt.-% comprising no nanoparticles.

TABLE 2
Shrinkage and conversion (DSC) of mixtures of nanoparticles

| | 1/bis- | 3/bis- | 5/bis- | BisGMA/ |
|---|---|---|---|---|
| Nanocomposit | GMA | GMA | GMA | TGDMA |
| Shrinkage ΔD V [%] | 6.8 | 6.2 | 5.4 | 7.1 |
| Conversion p [%] (DSC) after | 77 | 69 | 68 | 88 |
| 4 min irradiation | | | | |

### EXAMPLE 7

### Cocondensation to Nanoparticles in Resin Mixture

41.65 g (70.6 mmol) of macromonomer 6a, 36.77 g (176.5 mmol) of tetraethoxysilane were homogeneously mixed with 46.05 g ethylacetate and 105.00 g of a resin mixture comprising 80 wt.-% of 2,7,7,9,15-pentamethyl-4,13-dioxo-3,14-dioxa-5,12-diazahexadecane-1,16-diyl-dimethacrylate (UDMA), 15 wt.-% of diethyleneglycol dimethacrylate (DGDMA) and 5 wt.-% of trimethylol propane trimethacrylate (TMPTMA). The resin mixture is stabilised with 0.1 wt.-% BHT. Afterwards, for cocondensation of macromonomer 6a and tetraethoxysilane to nanoparticles 17.13 g of a 3.6 wt.-% aqueous solution of hydrogen fluoride was added in one portion while stirring the mixture intensely. After 3 days stirring at room temperature 13.02 g (91.6 mmol) of anhydrous sodium sulphate were added. Stirring was continued for a further day. Afterwards, sodium sulphate was filtered off and ethyl acetate and ethanol was evaporated. Product was found to be a clear liquid of 5.00 Pas viscosity at 23° C. and with a refractive index n_{D}=1.4775 at 20° C.

### COMPARATIVE EXAMPLE 1

0.48 g (94 mmol) of macromonomer 6a and 48.94 g (235 mmol) of tetraethoxysilane were homogeneouously mixed with 60.5 mg BHT in 27.83 g acetone. Afterwards, for cocondensation of macromonomer 6a and tetraethoxysilane to nanoparticles 22.82 g of a 3.6 wt.-% aqueous solution of hydrogen fluoride was added in one portion while stirring the mixture intensely. After 3 days stirring at room temperature a small amount of white precipitate was filtered of and acetone and ethanol were evaporated. To remove all water the residue was dissolved with 100 ml Chloroform and evaporated again. This procedure was repeated for 4 times. Afterwards, the nanoparticles which are a clear solid were redispersed in 48.86 g chloroform and 113,98 g resin mixture of the same composition as described in Example 7. For redispersion to a slightly turbid solution the mixture was treated for 20 min with ultra sound. Afterwards, chloroform was evaporated to yield a slightly turbid liquid of 20.8 Pas viscosity at 23° C. and with a refractive index n_{D}=1.4778 at 20° C.

### COMPARATIVE EXAMPLE 2

A homogeneous resin mixture comprising 720.00 g (80 wt.-%) of 2,7,7,9,15-pentamethyl-4,13-dioxo-3,14-dioxa-5,12-diaza-hexadecane 1,16-diyl-dimethacrylate (UDMA), 135.09 g (15 wt.-%) of diethyleneglycol dimethacrylate (DGDMA) and 45.05 g (5 wt.-%) of trimethylol propane trimethacrylate (TMPTMA) was prepared and stabilised with 900 mg BHT. The viscosity of the mixture is 1.33 Pas at 23° C. and the refractive index n_{D}=1.4740 at 20° C.

TABLE 3
Comparison of Example 7 and comparative examples 1 and 2

| | Comparative | | | | Comparative | |
|---|---|---|---|---|---|---|
| | Example 1 | | Example 7 | | Example 2 | |
| Resin mixture | 100 | wt.-% | 70 | wt.-% | 70 | wt.-% |
| Nanoparticles | 0 | wt.-% | 30 | wt.-% | 30 | wt.-% |
| Molar ratio | | | 1:2.5 | | 1:2.5 | |
| macromonomer | | | | | | |
| 6a:tetraethoxy- | | | | | | |
| silane | | | | | | |
| Viscosity at | 1.33 | Pas | 5.00 | Pas | 20.8 | Pas |
| 23° C. | | | | | | |
| Refractive | 1.4740 | | 1.4775 | | 1.4778 | |
| index | | | | | | |
| at 20° C. | | | | | | |

| | | | |
|---|---|---|---|
| Appearance | clear | clear | turbid |
| | liquid | liquid | liquid |

### APPLICATION EXAMPLE 1

30.00 g of nanoparticles of Example 1 were homogeneously mixed with 70.00 g 2,2-Bis-[p-(2-hydroxy-3-methacryloyloxypropoxy)-phenyl]-propane, 0.30 g camphor quinone, 0.35 g dimethylaminomethyl benzoic acid ethyl ester and 0.10 g di-tert.-butyl cresol. To this mixture were added 300 g of a bariumalumo-silicate glass mixed homogeneously. The composite is characterised by the following properties: compressive strength 255±34 MPa, flexural strength 68±9 MPa Young-lus modulus 1640±70 MPa.

### APPLICATION EXAMPLE 2

128.35 g of product of Example 7 were homogeneously mixed with 0.387 g camphorquinone, 0.452 g dimethylaminomethyl benzoic acid ethyl ester. To 100.00 g of this mixture were added 255 g of a strontium-fluoro-silicate glass and mixed homogeneously. The composite is characterised by the following properties: compressive strength 328±22 MPa, flexural strength 84±6 MPa, Young-modulus 6.27±0.37 GPa.

1. A process for the preparation of a polymerizable dental composition comprising the steps of
(a) preparing a liquid mixture comprising
(i) 1 to 99% w/w of a hybrid monomer component containing at least one hybrid monomer compound having one hydrolysable siloxane group and at least one polymerizable organic moiety, and
(ii) 99 to 1% w/w of a monomer component polymerizable with the polymerizable organic moiety of the hybrid monomer compounds; and
(b) adding at least a stoichiometrically sufficient amount of water to the mixture to hydrolyse the hydrolysable siloxane group of the hybrid monomer compound and to form spherical polymerizable nanoparticles having an average particle size of from 1 to 100 nm dispersed in the monomer component, whereby the nanoparticles have a structure with Si—O—Si bonds and peripherally exposed polymerizable organic moieties.

2. The process according to claim 1, wherein nanoparticles have an average particle size of from 1 to 20 nm.

3. The process according to claim 1, wherein nanoparticles have an average particle size of from 1 to 5 nm.

4. The process according to claim 1, wherein the hybrid monomer compound is a compound of the following formula (I) 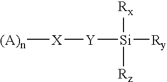
wherein
A is a polymerizable moiety, preferably an acrylate or methacrylate group;
Rₓ, R_{y}, R_{z}
which may be the same or different independently represent substituted or unsubstituted C₁ to C₁₈ alkoxy, C₅ to C₁₈ cycloalkoxy, a C₅ to C₁₅ aryloxy, C₂ to C₁₈ acyloxy or halogen;
X is a nitrogen atom or a substituted or unsubstituted C₁ to C₁₈ alkylene, C₁ to C₁₈ oxyalkylene or C₁ to C₁₈ carboxyalkylene group;
Y is a substituted or unsubstituted C₁ to C₁₈ alkylene, C₁ to C₁₈ oxyalkylene, C₅ to C₁₈ cycloalkylene, C₅ to C₁₈ oxycycloalkylene, C₅ to C₁₅ arylene, or C₅ to C₁₅ oxyarylene or heteroarylene group; and
n is an integer of 1 to 10.

5. The process according to claim 1, wherein the hybrid monomer compound is a compound of the following formulas 1-10: 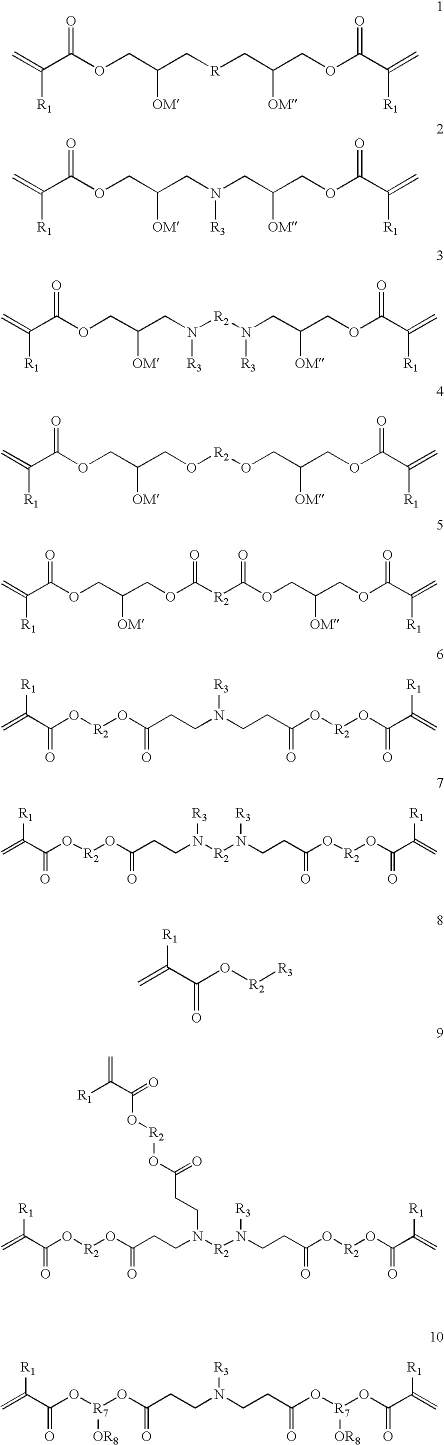
wherein
R is a residue derived from a diepoxide, notably a residue of the following formula 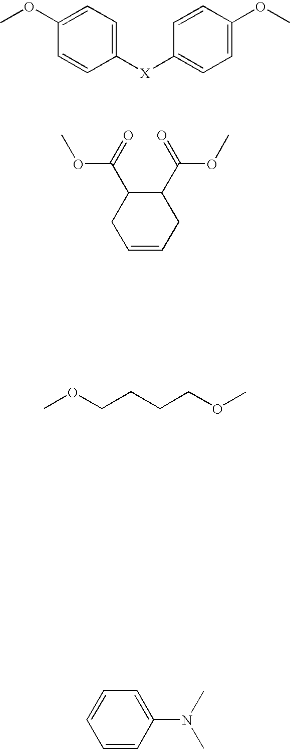
wherein X is C(CH₃)₂, —CH₂—, —O—, —S—, —CO—, or —SO₂—;
R₁ is hydrogen or a substituted or unsubstituted C₁ to C₁₈ alkyl, C₅ to C₁₈ cycloalkyl, C₅ to C₁₈ aryl or heteroaryl group;
R₂ is a divalent substituted or unsubstituted C₁ to C₁₈ alkylene, C₂ to C₁₂ alkenylene, C₅ to C₁₈ cycloalkylene, C₅ to C₁₈ arylene or heteroarylene,
R₃ which may represent the same or different substituents in formula 3 and 7, is a substituted or unsubstituted C₁ to C₁₈ alkyl, C₂ to C₁₂ alkenyl, C₅ to C₁₈ cycloalkyl, C₈ to C₁₂ aryl or C₇ to C₁₂ aralkyl group, or a siloxane moiety represented by one of the following formulae I, II or III 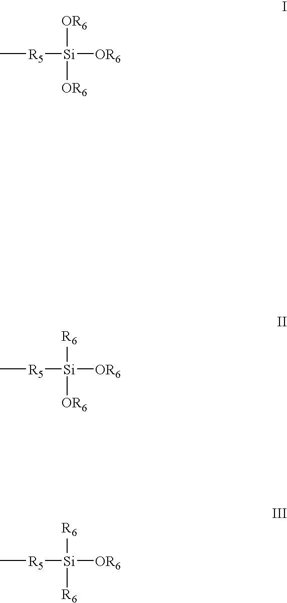
wherein
R₅ is a divalent substituted or unsubstituted C₁ to C₁₈ alkylene, C₂ to C₁₂ alkenylene, C₅ to C₁₈ cycloalkylene, C₅ to C₁₈ arylene or heteroarylene group, preferably CH₂CH₂CH₂,
R₆ is a substituted or unsubstituted C₁ to C₁₈ alkyl, C₂ to C₁₂ alkenyl, C₅ tO C₁₈ cycloalkyl, C₆ to C₁₂aryl or C₇ to C₁₂ aralkyl group,
R₇ is a substituted or unsubstituted C₁ to C₁₈ alkylene, C₂ to C₁₂ alkenyl, C₅ to C₁₈ cycloalkylene, C₅ to C₁₈ arylene or heteroarylene group,
R₈ is a protecting group for a hydroxyl group, preferably forming an ether, an ester or an urethane group,
M′ and M″
which may represent the same or different substituents, is a siloxane moiety represented by one of the following formulae IV, V or VI, a protecting group for a hydroxyl group, preferably forming an ether, an ester or an urethane group, or hydrogen in case R₃ is a siloxane moiety represented by one of formulae I, II, or III as defined above, 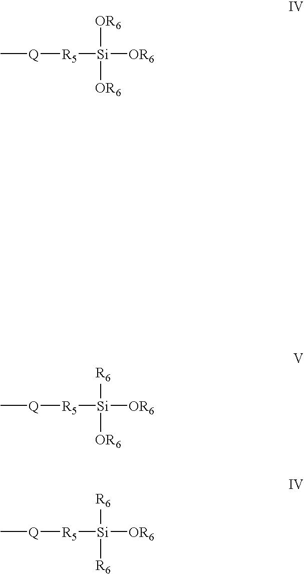
wherein
Q is an ether, an ester, a urethane or thiourethane linking group, and
R₅ and R₆ are as defined above.

6. The process according to claim 1, wherein the hybrid monomer component comprises a compound of the following formula 11 or 12:

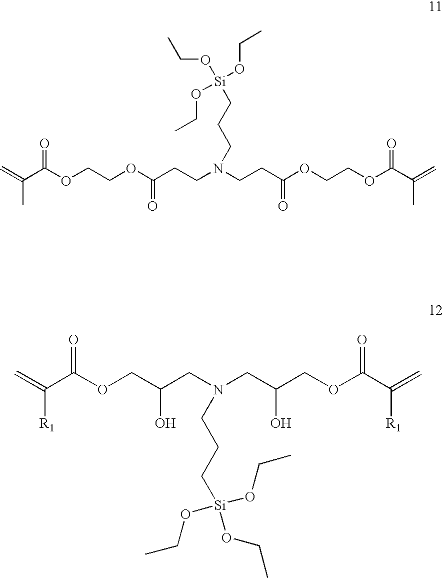

7. The process according to claim 1, wherein said polymerizable monomer is a mono- or polyfunctional acrylate or methacrylate, selected from the group of methyl methacrylate, ethyleneglycol dimethacrylate diethyleneglycol dimethacrylate triethyleneglycol dimethacrylate, 3, (4),8, (9)-dimethacryloyloxymethyltricyclodecane, dioxolan bismethacrylate, vinyl-, vinylen- or vinyliden-, acrylic- or methacrylic substituted spiroorthoesters, spiroorthocarbonates or bicyloorthoesters, glycerin trimethacrylate, trimethylol propane triacrylate, furfurylmethacrylate.

8. The process according to claim 1, wherein the nanoparticles are formed in the presence of metal compounds selected from the group of alkoxides or metal complexes such as metal acetyl acetonates whereby the metals are selected from the group of Ba, Al, La, Ti, Zr, TI, In or other transition elements or elements of the lanthanides or actinides.

9. The process according to claim 1, further comprising the step of adding an inorganic filler selected from La₂O₃, ZrO₂, BiPO₄, CaWO₄, BaWO₄, SrF₂, Bi₂O₃, a porous glass or an organic filler, such as polymer granulate, embrittled glass fibres or a combination of organic and/or inorganic fillers or reactive inorganic fillers.

10. The process according to claim 1, further comprising the step of adding a polymerisation initiator and a stabiliser.

11. The process according to claim 1, wherein hydrolysis is carried out in the presence of a catalyst.

12. The process according to claim 12, wherein the catalyst is an acid or base.

13. The process according to claim 1, wherein hydrolysis is carried out under neutral conditions.

14. The process according to claim 1, wherein the composition comprises a polymerizable di- or poly(meth)acrylate, at least a polymerizable monomer, polymerisation initiators and/or sensitisers and stabilisers.

15. The process according to claim 1, wherein hydrolysis is carried out in the presence of an organic solvent such as THF, dioxane, chloroform, toluene, acetone.

16. The process according to claim 1, wherein hydrolysis is carried out in the presence of polymerizable monomers such as methyl methacrylate, ethylene glycol dimethacrylate, diethylene glycol dimethacrylate, triethylene glycol dimethacrylate, trimethylol propane triacrylate, 3,(4),8, (9)-dimethacryloyloxymethyltricyclo decane, dioxolan bismethacrylate, glycerol trimethacrylate, furfuryl methacrylate.

17. A polymerizable dental composition obtainable according to the process of any one of claim 1.
